(19) United States
(12) Patent Application Publication
(10) Pub. No.: US 2006/0212037 A1
(43) Pub. Date: Sep. 21, 2006

(54) Pumping chamber for a liquefaction handpiece

(75) Inventors: Glenn Sussman, Laguna Nigel, CA (US); John R. Underwood, Laguna Nigel, CA (US)

Correspondence Address: ALCON, IP LEGAL, TB4-8, 6201 SOUTH FREEWAY, FORT WORTH, TX, 76134, US

(73) Assignee: ALCON, INC.

(21) Appl. No.: 11/081,460

(22) Filed: Mar. 16, 2005

(51) Int. Cl. A61F 9/00

(52) U.S. Cl. 606/107

(57) ABSTRACT

A liquefaction surgical handpiece having a capacitance chamber upstream of the boiling chamber.

### BACKGROUND OF THE INVENTION

This invention relates generally to the field of ophthalmic and otic surgery and more particularly to a pumping chamber for a handpiece for ophthalmic and otic surgery.

The human eye in its simplest terms functions to provide vision by transmitting light through a clear outer portion called the cornea, and focusing the image by way of the lens onto the retina. The quality of the focused image depends on many factors including the size and shape of the eye, and the transparency of the cornea and lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

A typical ultrasonic surgical device suitable for ophthalmic procedures consists of an ultrasonically driven handpiece, an attached cutting tip, and irrigating sleeve and an electronic control console. The handpiece assembly is attached to the control console by an electric cable and flexible tubings. Through the electric cable, the console varies the power level transmitted by the handpiece to the attached cutting tip and the flexible tubings supply irrigation fluid to and draw aspiration fluid from the eye through the handpiece assembly.

The operative part of the handpiece is a centrally located, hollow resonating bar or horn directly attached to a set of piezoelectric crystals. The crystals supply the required ultrasonic vibration needed to drive both the horn and the attached cutting tip during phacoemulsification and are controlled by the console. The crystal/horn assembly is suspended within the hollow body or shell of the handpiece by flexible mountings. The handpiece body terminates in a reduced diameter portion or nosecone at the body's distal end. The nosecone is externally threaded to accept the irrigation sleeve. Likewise, the horn bore is internally threaded at its distal end to receive the external threads of the cutting tip. The irrigation sleeve also has an internally threaded bore that is screwed onto the external threads of the nosecone. The cutting tip is adjusted so that the tip projects only a predetermined amount past the open end of the irrigating sleeve. Ultrasonic handpieces and cutting tips are more fully described in U.S. Pat. Nos. 3,589,363; 4,223,676; 4,246,902; 4,493,694; 4,515,583; 4,589,415; 4,609,368; 4,869,715; 4,922,902; 4,989,583; 5,154,694 and 5,359,996, the entire contents of which are incorporated herein by reference.

In use, the ends of the cutting tip and irrigating sleeve are inserted into a small incision of predetermined width in the cornea, sclera, or other location. The cutting tip is ultrasonically vibrated along its longitudinal axis within the irrigating sleeve by the crystal-driven ultrasonic horn, thereby emulsifying the selected tissue in situ. The hollow bore of the cutting tip communicates with the bore in the horn that in turn communicates with the aspiration line from the handpiece to the console. A reduced pressure or vacuum source in the console draws or aspirates the emulsified tissue from the eye through the open end of the cutting tip, the cutting tip and horn bores and the aspiration line and into a collection device. The aspiration of emulsified tissue is aided by a saline flushing solution or irrigant that is injected into the surgical site through the small annular gap between the inside surface of the irrigating sleeve and the cutting tip.

Recently, a new cataract removal technique has been developed that involves the injection of hot (approximately 45° C. to 105° C.) water or saline to liquefy or gellate the hard lens nucleus, thereby making it possible to aspirate the liquefied lens from the eye. Aspiration is conducted with the injection of the heated solution and the injection of a relatively cool solution, thereby quickly cooling and removing the heated solution. This technique is more fully described in U.S. Pat. No. 5,616,120 (Andrew, et al.), the entire contents of which is incorporated herein by reference. The apparatus disclosed in the publication, however, heats the solution separately from the surgical handpiece. Temperature control of the heated solution can be difficult because the fluid tubings feeding the handpiece typically are up to two meters long, and the heated solution can cool considerably as it travels down the length of the tubing.

One liquefaction handpiece, generally described in U.S. Pat. Nos. 5,989,212, 6,575,929 B2, and 6,676,628 B2 (all to Sussman, et al.) and commercially available as the AUQALASE® handpiece from Alcon Laboratories, Inc., Fort Worth, Tex., contains an internal boiling chamber. The pulse repetition rate of this handpiece is less than optimal because of the time required to refill the boiling chamber between pulses. The entire contents of these patents are incorporated herein by reference, specifically column 3, lines 47-67, column 4, lines 1-32 and FIGS. 7 and 8 of U.S. Pat. No. 5,989,212, column 3, lines 40-67, column 4, lines 1-32 and FIGS. 7 and 8 of U.S. Pat. No. 6,575,929 and column 3, lines 47-67, column 4, lines 1-37 and FIGS. 7 and 8 of U.S. Pat. No. 6,676,628.

Therefore, a need continues to exist for a control system for a surgical handpiece that can more rapid pulses of heated solution used to perform liquefaction surgical procedures.

### BRIEF SUMMARY OF THE INVENTION

The present invention improves upon the prior art by providing a liquefaction surgical handpiece having a capacitance chamber upstream of the boiling chamber.

Accordingly, one objective of the present invention is to provide a surgical handpiece having a pumping chamber with two electrodes.

Another objective of the present invention is to provide a surgical handpiece having a device for delivering the surgical fluid through the handpiece in rapid pulses.

Another objective of the present invention is to provide a liquefaction surgical handpiece having a capacitance chamber upstream of the pumping chamber.

These and other advantages and objectives of the present invention will become apparent from the detailed description and claims that follow.

### BRIEF DESCRIPTION OF THE DRAWINGS

FIG. 1 is a perspective view of a surgical system that may be used with the handpiece of the present invention.

FIG. 2 is a partial cross-sectional view of the handpiece of the present invention.

### DETAILED DESCRIPTION OF THE INVENTION

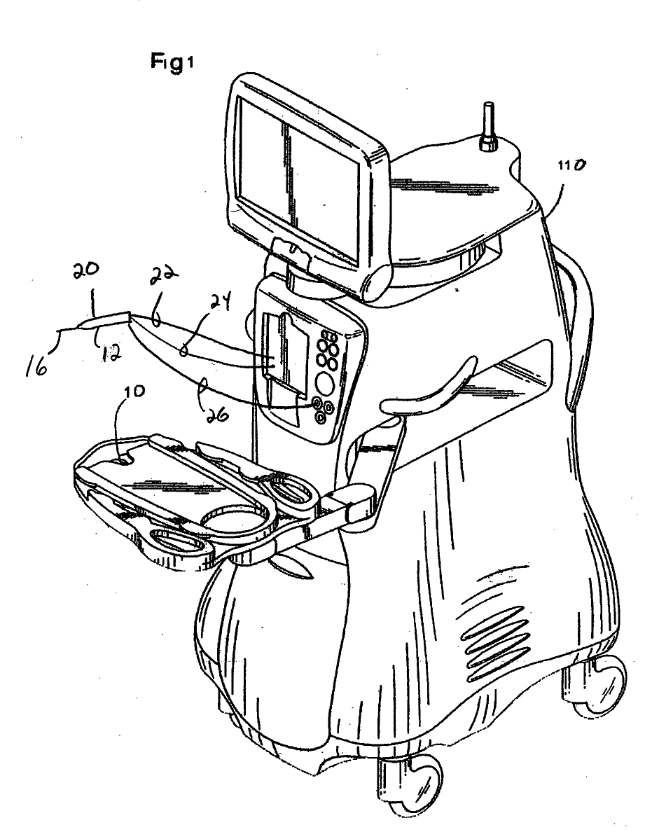

As best seen in FIG. 1, commercially available surgical systems generally include surgical console 110 having attached mayo tray 10 and handpiece 20 attached to console 110 by aspiration tubing 22, irrigation tubing 24 and power cable 26. Power to handpiece 20 as well as flows of irrigation and aspiration fluids are controlled by console 110, which contains appropriate hardware and software, such as power supplies, pumps, pressure sensors and valves, all of which are well-known in the art.

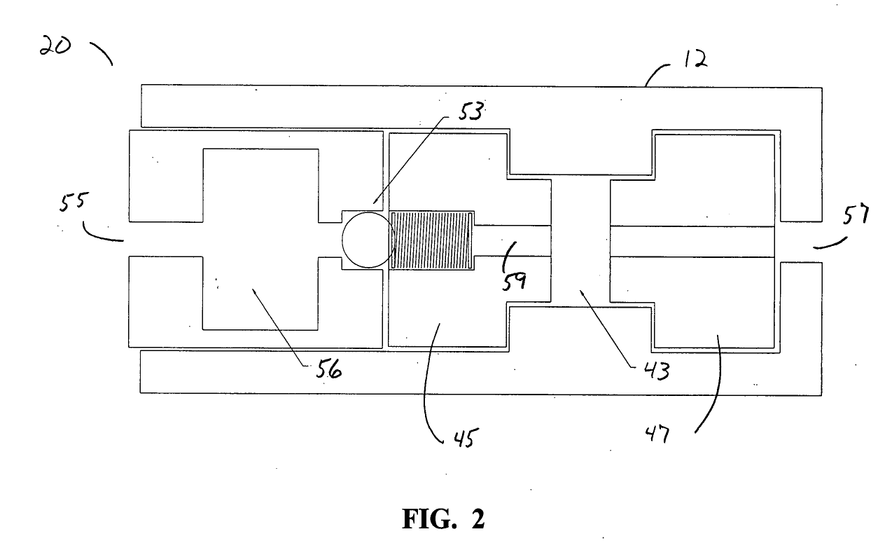

Handpiece 20 of the present invention generally includes handpiece body 12 and operative tip 16. Contained within body 12, as best seen in FIG. 2, are proximal electrode 45 and distal electrode 47 which define pumping reservoir 43. Electrical power is supplied to electrodes 45 and 47 by insulated wires, not shown. In use, surgical fluid (e.g. saline irrigating solution) enters reservoir 43 through port 55, capacitance chamber 56, check valve 53 and inlet 59, check valves 53 being well-known in the art. Electrical current (preferably Radio Frequency Alternating Current or RFAC) is delivered to and across electrodes 45 and 47 because of the conductive nature of the surgical fluid. As the current flows through the surgical fluid, the surgical fluid boils. As the surgical fluid boils, it expands rapidly out of pumping chamber 43 through port 57 (check valve 53 prevents the expanding fluid from entering capacitance chamber 56). The expanding gas bubble pushes the surgical fluid in port 57 downstream of reservoir 43 forward. Subsequent pulses of electrical current form sequential gas bubbles that move surgical fluid out port 57. The size and pressure of the fluid pulse obtained out of reservoir 43 can be varied by varying the length, timing and/or power of the electrical pulse sent to electrodes 45 and 47 and by varying the dimensions of reservoir 43.

The repetition rate of the pulses generated in reservoir 43 are limited by the amount of time it take to refill reservoir 43 after a pressurized pulse has been discharge out of port 57. Many factors can affect this refill time, including resistance in irrigation tubing 24, which may be the source of fluid for reservoir 43. Placing the irrigation fluid source closer to reservoir 43 will reduce this resistance, but incorporating a fluid source large enough for a complete surgical procedure into handpiece 20 may not be desirable. Therefore, handpiece 20 of the present invention incorporates capacitance chamber 56 within handpiece 20 and very near reservoir 43. Capacitance chamber 56 contains a sufficient quantity, approximately between 4 and 100 microliters, of surgical fluid for several pressure pulses and due to the proximity of capacitance chamber 56 to reservoir 43, allows for more rapid refilling of reservoir 43.

This description is given for purposes of illustration and explanation. It will be apparent to those skilled in the relevant art that changes and modifications may be made to the invention described above without departing from its scope or spirit. For example, it will be recognized by those skilled in the art that the present invention may be combined with ultrasonic and/or rotating cutting tips to enhance performance.

We claim:

1. A handpiece, comprising:
a) a body having an integral pumping reservoir, the pumping reservoir having an inlet and being defined by a pair of electrodes; and
b) a capacitance chamber integrally formed within the body and fluidly connected to the inlet of the pumping reservoir, the capacitance chamber being sized so as to contain a sufficient quantity of surgical fluid for several pressure pulses from the pumping chamber.

2. The handpiece of claim 1 wherein the pumping reservoir and the capacitance chamber are fluidly connected through a check valve.
